(19) United States
(12) Patent Application Publication
(10) Pub. No.: US 2009/0104171 A1
(43) Pub. Date: Apr. 23, 2009

(54) Metabolic Enhancement Therapy

(76) Inventors: Joel D. Pardee, Patterson, NJ (US); Alexander V. Birk, Doylestown, PA (US)

Correspondence Address: FOX ROTHSCHILD LLP, 2000 MARKET STREET, 10th Floor, PHILADELPHIA, PA, 19103, US

(21) Appl. No.: 12/046,120

(22) Filed: Mar. 11, 2008

Related U.S. Application Data
(60) Provisional application No. 60/981,246, filed on Oct. 19, 2007.

(51) Int. Cl. A61K 31/4188; A61P 25/00; A61P 25/18

(52) U.S. Cl. 424/94.1

(57) ABSTRACT

A Metabolic Enhancement Therapy nutritional supplement for treating symptoms and conditions such as acute anxiety, hyperactivity, panic attacks, confusion, involuntary movements, sleep disorders, mood disorders, depression, anger, seizures, obsessive-compulsive behavior, unregulated fear, panic, neuronal disorders, autism, schizophrenia, attention deficit disorder (ADD), attention deficit hyperactive disorder (ADHD) and neuronally based immunologic disorders by administering to a mammal an effective amount of acetyl-L-carnitine, α-lipoic acid, biotin, vitamin B1, L-glutamine, Vitamin B6, taurine, folinic acid, vitamin B12, trimethylglycine, and L-tryptophan.

### CROSS-REFERENCE TO RELATED APPLICATIONS

This application is based on and claims priority to U.S. Provisional Application No. 60/981,246, filed Oct. 19, 2007, the entire contents of which are hereby incorporated by reference.

### BACKGROUND OF THE INVENTION

### Field of the Invention

The present invention is directed to nutritional supplements used to provide a balance of excitatory and inhibitory neurotransmitters to the human brain. The supplements and methods of the present invention may be used to treat a variety of conditions which include symptoms of acute anxiety, hyperactivity, confusion, involuntary movements, insomnia, obsessive/compulsive behaviors, depression, anger, and panic often associated with the neuronal disorders including autism, attention deficit disorders, schizophrenia and neuronally based immunologic disorders.

The present invention may be particularly useful in treating autism. Autism is a developmental neuronal disorder that is reaching alarming epidemic proportions in the United States and elsewhere around the world. The latest demographic statistics indicate that approximately 1 in 150 children are affected nationwide, with a 2 to 5:1 ratio of boys affected compared to girls. The disorder, once considered restricted to early childhood, is now known to persist throughout adult life. The molecular cause of autism is unknown, there is no known cure, and although recent efforts to deliver pharmaceuticals have been reported and patented, none have proved effective in ameliorating and overcoming the multiplicity of neurologic and behavioral symptoms endemic to the disease. Afflicted individuals exhibit a wide spectrum of behavioral patterns and intellectual developments depending on their degree of affliction. Consequently, autistics comprise persons of intellectual genius and those with significant mental retardation, persons with developed language skills (Asberger's Syndrome) to those who cannot communicate with the spoken word, and behaviorally normal individuals to those who are deeply emotionally disturbed. The disease can include acute language disorders, echolalia, mental confusion, inattention, lack of intellectual focus, general lassitude, obsessive/compulsive behavior, paranoia, acute anxiety, panic attacks, seizures, low motile bowel syndrome, explosive diarrhea, physical aggression, self-mutilation, uncontrolled involuntary movements, depression, mania (hyperactivity), agoraphobia, nutrient malabsorption, compromised immune function, and acute acoustic sensitivity. Many of the neurologic and behavioral deficits are also apparent in persons diagnosed with clinical depression, schizophrenia, bipolar disorder, and attention deficit disorder (ADD), for which clear genetic correlates have not been found.

Symptomatic similarities for the variety of neurologic disorders discussed above suggest a common generic root of functional imbalance between excitatory and inhibitory neuron function. The theory of imbalanced excitatory/inhibitory neuron function in the brain points to a crucial need to find therapeutic interventions to redress imbalanced neuron function. The present invention is directed to “Metabolic Enhancement” to normalize inhibitory and excitatory neuron function. In particular, the present invention is a form of “Metabolic Enhancement Therapy” that stimulates specifically targeted neuronal biochemical pathways with non-toxic, naturally occurring metabolites, vitamins and enzyme cofactors to achieve balanced neuronal transmitter synthesis. The compositions and methods of the present invention can be used to effectively treat a wide variety of disease states in addition to the neurological conditions discussed above, including, but not limited to: immunologic deficiency, glucose metabolism, cellular ageing, smooth and skeletal muscle disorders, intestinal malabsorption, physiologic and psychologic stress syndromes, migraine headache, wound healing, obesity, cell hyperproliferation in benign and malignant tumor growth, chronic fatigue syndrome, arthritis, asthma, and childhood development disorders. The present invention may also be used to treat fundamental metabolic issues prevalent in a number of non-neuronal pathologies that are currently incurable.

The three major excitatory neuronal pathways described in the brain are the dopaminergic, noradrenergic, and cholinergic pathways. Each of these are modulated by inhibitory glycinergic, serotinergic, and gamma amino butyric acid (GABAergic) neurons. Which inhibitory pathway is utilized to modulate any given excitatory pathway depends on brain location and excitatory neuron function. Based on the Metabolic Enhancement Therapy model of the present invention, each of the metabolic pathways leading to synthesis of the inhibitory neural transmitters; glycine, serotonin, and GABA, are regulated in general by mitochondria-dependent cellular energy state together with appropriate metabolic pathways that can be stimulated naturally with appropriate enzyme substrates and cofactors. Using Metabolic Enhancement Therapy, toxic effects are effectively eliminated, because metabolic balance is achieved through selective stimulation of metabolic pathways that are under-producing desired neurotransmitters, and because normally functioning pathways are not overstimulated when provided with naturally occurring metabolites. This selective stimulation of only those pathways that are suboptimally functional allows for safe administration of the supplement of the present invention to subjects possessing the widely varying metabolisms thought to be characteristic of different forms of neuronal disorders.

### SUMMARY OF THE INVENTION

The present invention is directed to a variety of supplements and methods which may be used to treat or prevent a variety of diseases and conditions. One embodiment of the present invention is a nutritional supplement comprising: acetyl-L-carnitine, α-lipoic acid, biotin, vitamin B1, L-glutamine, Vitamin B6, taurine, folinic acid, vitamin B12, trimethylglycine, and L-tryptophan.

In another embodiment, the supplement further comprises at least one ingredient selected from the group consisting of lithium orotate, vitamin C, vitamin E, vitamin D, vitamin B2 (riboflavin), vitamin B3 (niacin), Nystatin, vitamin B5 (pantothenic acid), calcium and magnesium.

In yet another embodiment, the supplement further comprises vitamin B5 (pantothenic acid), vitamin B3 (niacin), vitamin B2 (riboflavin).

The supplements of the present invention may be in the form of a liquid, powder, or combination of liquid and powder.

Suitable forms of the supplement include a liquid formulation suitable for oral administration, a capsule form, and a powder suitable for sprinkling on food.

The supplement of the present invention may also be dissolved in a liquid, present in a sustained release gel or provided as a patch.

The supplement of the present invention may also be in a form suitable for injection.

The supplements of the present invention may be used in a variety of methods, including a method to normalize inhibitory and excitatory neuron function in a mammal comprising administration to a mammal a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.

The invention also provides a method to modify mitochondrial metabolism comprising administration to a mammal a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.

The present invention also provides a method for preventing cellular accumulation of homocysteine comprising administration to a mammal a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.

In addition, the present invention includes a method for increasing metabolic production of serotonin, glycine and GABA in a mammal comprising administering to the mammal a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.

The invention also provides methods for treating a variety of diseases and conditions. Accordingly, the present invention includes a method for treating a mammal having a disease or condition selected from acute anxiety, hyperactivity, panic attacks, confusion, involuntary movements, a sleep disorder, a mood disorder, depression, anger, seizures, obsessive-compulsive behavior, unregulated fear, panic, or attention deficit disorder, comprising administering to the mammal a nutritional supplement comprising acetyl-L-carnitine, α-lipoic acid, biotin, vitamin B1, L-glutamine, Vitamin B6, taurine, folinic acid, vitamin B12, trimethylglycine (betaine), and L-tryptophan.

Another method involves treating a mammal having a disease selected from autism, schizophrenia attention deficit disorder (ADD), attention deficit hyperactive disorder (ADHD) and neuronally based immunologic disorders comprising administering to the mammal a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.

The present invention also provides a method for treating physical hyperactivity, involuntary movements, low motile bowel syndrome, or intestinal malabsorption in a mammal comprising administering to the mammal a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.

### BRIEF DESCRIPTION OF THE DRAWINGS

FIG. 1. Emotional Balance through Metabolic Enhancement Therapy. FIG. 1 is a diagram illustrating the interactions involved in Metabolic Enhancement Therapy.

### DETAILED DESCRIPTION OF THE PREFERRED EMBODIMENTS

The present invention is directed to nutritional supplements which may be used to treat a variety of disorders which are based in part on the balance of excitatory and inhibitory neurotransmitters. These supplements may modify both cellular and mitochondrial metabolism, prevent cellular accumulation of homocysteine, and modify the metabolic production of serotonin, glycine, and GABA.

In particular, the present invention relates to a supplement formulation which includes folinic acid, trimethylglycine (betaine), acetyl L-carnitine, α-lipoic acid, biotin, L-glutamine, L-tryptophan, taurine, vitamins B1, B6, and B12. This supplement may metabolically enhance mitochondria-derived energy, protect mitochondria against harmful effects of homocysteine, and stimulate the energy-dependent synthesis in brain of the essential inhibitory neurotransmitters glycine, serotonin and gamma amino butyric acid (GABA).

Additional forms of the supplement may include lithium orotate, vitamin C, vitamin E, vitamin B2 (riboflavin), vitamin B3 (niacin), Nystatin (prescription), vitamin B5 (pantothenic acid), calcium, and magnesium taken in conjunction with the basic supplement formulation to therapeutically address excessive mood swings, enhance mitochondrial energy charge, inhibit mitochondrial oxidative stress and stimulate growth of bone and muscle. Administration of the compositions of the present invention may involve increasing the metabolic rate of brain cells of a mammalian host with respect to metabolic production of ATP in general support of neural transmitter synthesis. In particular, acetyl-L-carnitine may promote acetyl CoA production from neuron mitochondrial P-oxidation. L-tryptophan is a metabolic precursor of serotonin and melatonin; vitamin B6 is a required cofactor for serotonin and glycine synthesis; vitamin B12 is a required cofactor for conversion of homocysteine to methionine by activated folic acid; trimethylglycine (TMG) is a required substrate for methylation of homocysteine to methionine and biosynthesis of glycine; L-glutamine is an easily absorbed substrate for synthesis of GABA; taurine is an easily absorbed inhibitory neurotransmitter, folinic acid is a required cofactor for synthesis of glycine, methionine and S-adenosylmethionine; α-lipoate and biotin are required for optimal conversion of pyruvate to acetyl-CoA in TCA cycle energy production. Folinic acid, vitamin B12, and TMG also protect against mitochondrial oxidative stress resulting from the accumulation of homocysteine and diminution of glutathione. These various ingredients are discussed in detail below along with their potential role in Metabolic Enhancement Therapy.

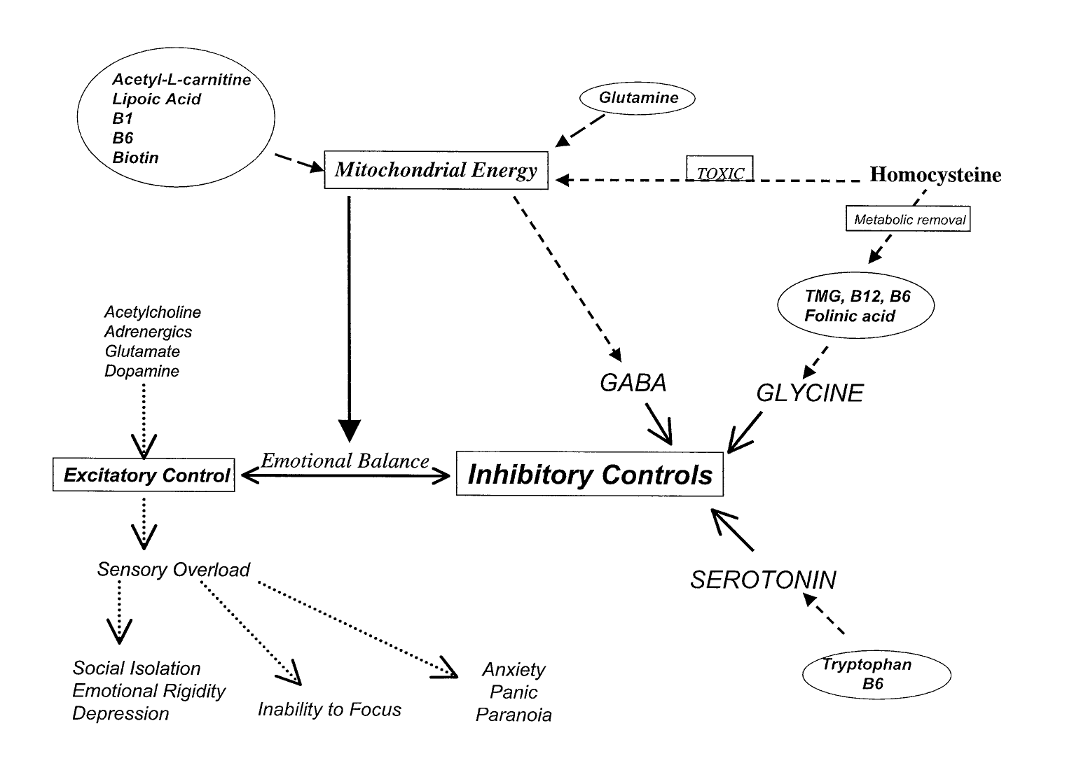

An overview of the interactions involved in Metabolic Enhancement Therapy is provided in FIG. 1. Referring now to FIG. 1, emotional and psychological balance of human social behavior is based primarily on balanced interplay between excitatory and inhibitory neurons in a brain [Emotional Balance]. Excitatory stimulations via excitatory nerves are initiated by the excitatory neurotransmitters such as acetylcholine, adrenergics, glutamate and dopamine [Excitatory Control]. Unchecked and uninhibited excitatory nerve activation leads to sensory overload that expresses itself in overt psychological problems such as acute anxiety, hyperactivity, social isolation, emotional rigidity, obsessive/compulsive behaviors, depression, anger, panic, paranoia, insomnia, involuntary movements and an inability to intellectually focus [Sensory Overload]. These psychological problems are commonly observed in a number of common neuronal disorders such as autism, attention deficit disorder, schizophrenia and neuronal-based immunologic disorders. To balance excitation, an equivalent activation of inhibitory neurons that interact directly with excitatory neurons, is required [Inhibitory Control]. In normal metabolism, it is a primary role of the inhibitory neuron neurotransmitters, [GABA, GLYCINE, and SEROTONIN], to offset and balance excitatory inputs, generating emotional and behavioral balance [Emotional Balance]. The balance between excitatory and inhibitory components is tightly regulated by metabolism, and depends on cellular energy levels maintained primarily by mitochondria [Mitochondrial Energy]. In the context of neuronal regulation, optimized mitochondrial energy is required to synthesize GABA from ingested glutamine, SEROTONIN from ingested tryptophan, and GLYCINE from a variety of ingested amino acids. All of these metabolic biosynthetic pathways require the presence of adequate amounts of energy, coenzymes and cofactors to drive inhibitory neurotransmitter synthesis. In addition, optimized mitochondrial metabolism is required to produce GABA from glutamine, serotonin from tryptophan, glycine, where if deprived, mitochondria would simply burn glutamine, tryptophan and glycine to produce energy in expense of synthesizing relevant inhibitory neurotransmitters. In Metabolic Enhancement Therapy, proper coenzymes, cofactors and starting materials are supplied by pure nutritional supplements to enhance synthesis of GABA, GLYCINE and SEROTONIN. Thus, to optimize mitochondria metabolism the present invention uses an energy component of the formula, which includes acetyl-L-carnitine, α-lipoic acid, vitamin B1, vitamin B6, biotin and glutamine. Vitamin B6 in our formula is also required to promote synthesis of serotonin from supplemented tryptophan. Supplied Glutamine, in addition to optimizing mitochondria metabolism, is a substrate for GABA production. The rest of the formula, TMG, vitamin B12, and Folinic acid, which also includes vitamin B6, is required for synthesis of glycine, in addition to removing the cellular toxin, Homocysteine. Taken together, the combination of nutritional supplements provided by the disclosed Metabolic Enhancement Therapeutic act in concert to effectively produce emotional balance and release individuals from the debilitating effects of neuronal dysfunction.

Ingredients which may be used in the supplements of the invention are discussed below along with their possible activity.

### Optimization of Mitochondrial Energy Production (Acetyl-L Carnitine, α-Lipoic Acid, Biotin, Vitamin B1)

In neurons mitochondria must function optimally to provide necessary ATP energy to sustain normal neurotransmitter (NT) synthesis.

Acetyl-L-carnitine is administered to optimize fatty acid transport into mitochondria, stimulating beta-oxidation of imported palmityl CoA to provide FADH derived ATP, and acetyl CoA to enter the tricarboxylic acid (TCA) cycle and generate NADH derived ATP. Beta-oxidation of fatty acids is the predominant source of Acetyl CoA in mitochondria. Acetyl-L-carnitine, known to be taken into mitochondria via transporters, is metabolized to acetate and carnitine. Imported acetate is then directly utilized to form acetyl CoA, and carnitine is used to transport fatty acids in the form of palmityl CoA into the mitochondria to form additional acetyl-CoA and ATP. Preliminary trials have found that acetyl-L-carnitine supplementation was effective in relieving depression in a group of elderly people with serious clinical symptoms, suggesting that mitochondrial metabolism and ATP generation in severely depressed patients is likely to play a significant role in regulation of serotonin synthesis. (Tempesta E et al. Drugs Exp Clin Res 1987; 13:417-23.; Garzya G, et al. Drugs Exp Clin Res 1990; 16:101-6.; Guarnaschelli C, et al. Drugs Exp Clin Res 1988; 14:715-8.; Bella R, et al. Int J Clin Pharmacol Res 1990; 10:355-60.).

Carnitine and carnitine derivatives have been used as supplements in animal husbandry, and for human diet and therapy as described in the following patents: [U.S. Pat. No. 5,362,753 (Method of increasing the hatchability of eggs by feeding hens carnitine); U.S. Pat. No. 4,687,782 (Nutritional composition for enhancing skeletal muscle adaptation to exercise training); U.S. Pat. No. 5,030,458 (Method for preventing diet-induced carnitine deficiency in domesticated dogs and cats); U.S. Pat. No. 5,030,657 (L-carnitine supplemented catfish diet); U.S. Pat. No. 4,343,816 (Pharmaceutical composition comprising an acyl-carnitine, for treating peripheral vascular diseases); U.S. Pat. No. 5,560,928 (Nutritional and/or dietary composition and method of using the same); U.S. Pat. No. 5,504,072 (Enteral nutritional composition having balanced amino acid profile); U.S. Pat. No. 5,391,550 (Compositions of matter and methods for increasing intracellular ATP levels and physical performance levels and for increasing the rate of wound repair); U.S. Pat. No. 5,240,961 (Method of treating reduced insulin-like growth factor and bone loss associated with aging).] Acetyl-L-carnitine rather than L-carnitine is preferred in the supplements of the present invention because of its highly preferred property of easy access to the brain through the blood brain barrier.

α-Lipoic Acid. α-Lipoate is required for optimal conversion of pyruvate to acetyl-CoA in TCA cycle energy production. α-Lipoate is a coenzyme that is essential to the function of pyruvate dehydrogenase to convert pyruvate to acetyl CoA, and to α-ketoglutarate dehydrogenase for conversion of α-ketoglutarate to succinate. Both reactions occur in mitochondria to turn the TCA cycle which generates ATP and cell energy. Fully functional pyruvate dehydrogenase is absolutely essential for energy production from carbohydrates. It is therefore found in high concentrations in the mitochondrial compartment of all tissues, including brain. It achieves full functionality only by binding 5 separate coenzymes or prosthetic groups, vitamin B1 (thiamine), α-lipoic acid, coenzyme A, flavin adenine dinucleotide (FAD) and nicotinamide adenine dinucleotide (NAD), that must also be present in high concentrations matching that of the enzyme. A variety of disorders in pyruvate metabolism have been detected in children, often involving deficiencies in the pyruvate dehydrogenase multienzyme complex. Altered forms of various subunits of the enzyme can result in reduced binding of the above described coenzymes or prosthetic groups, resulting in sub-optimal function of the enzyme and resulting deficiencies in energy production. Children detected with a pyruvate dehydogenase functional deficiency frequently exhibit neurological defects, with severe deficiency resulting in death. In less severe cases patients respond positively to dietary management, which includes minimizing carbohydrate intake and enhanced nutritional supplementation with the essential coenzymes and prosthetic groups required for pyruvate dehydrogenase function.

Biotin. Biotin is the prosthetic group for a number of carboxylation reactions, including synthesis of oxaloacetate for replenishment of the TCA cycle, and for biosynthesis of fatty acids via acetyl CoA for fatty acid transport into mitochondria and energy production by beta-oxidation. Biotin is normally synthesized in more than adequate amounts by intestinal bacteria, but can become deficient if the gut flora is disturbed by long-term antibiotic treatment, chronic yeast infections, or malabsorption, conditions that are often associated with autism.

Vitamin B1. Vitamin B1 (Thiamine) is rapidly converted in the body to thiamine pyrophosphate (TPP), which is required for key reactions catalyzed by pyruvate dehydrogenase and α-ketoglutarate dehydrogenase in mitochondria. Consequently, the cellular capacity for energy production is severely compromised in thiamine deficiency. TPP is also required for synthesis of ribose from the pentose phosphate pathway, which is absolutely required for synthesis of nucleic acid precursors for RNA and DNA, and it is the major source of NADPH for fatty acid and other biosynthetic pathways. Finally, TPP plays an important role in transmission of nerve impulses. TPP localized in peripheral nerve membranes is required for synthesis of the neurotransmitter acetylcholine absolutely required for neuromuscular activation of muscle contraction. Thiamine deficiency appears to selectively inhibit energy production from carbohydrates, and compromise muscle function. Loss of appetite, constipation, nausea, mental depression, peripheral neuropathy, irritability and chronic fatigue characterize thiamine deficiency. These symptoms are most often seen in the elderly, in poor persons on restricted diets, and in neural disorders such as autism and schizophrenia. Thiamine requirement is proportional to the caloric content of the diet and has been determined to be in the range of 1.4-1.5 mg per day for a normal adult. The requirement is raised if carbohydrate intake is excessive. Vitamin B1 is readily excreted if its concentration surpasses the renal threshold. Thus toxicities are very rare.

### GABA Synthesis (L-Glutamine, Vitamin B6, Taurine)

Gamma amino butyric acid (GABA), is a major inhibitory neurotransmitter (NT) which emanates from gabaergic neurons. Closely related glutamatergic neurons that can fulfill either an excitatory or inhibitory function secrete glutamate as the NT, and are in turn acted upon by extracellular glutamate. Gabaergic neurons are known to modulate excitatory neurons in the brain and spinal cord that trigger involuntary movements, as well as to inhibit excitatory neurotransmission in the brain that hamper the ability to mentally focus and concentrate. Clearly, inhibition of involuntary movement is desirable in autism, as is enhancing the ability to concentrate and productively lengthen attention span. Recent research implicates gabaergic neuron dysfunction in autism via folic acid polymorphisms that directly affect the kainite receptors on this class of neuron (Ruzicka et al., “Selective epigenetic alteration of layer I GABAergic neurons isolated from prefrontal cortex of schizophrenia patients using laser-assisted microdissection,” Mol. Psychiatry 12(4):385-97 (April, 2007); Veldic et al., “Epigenetic mechanisms expressed in basal ganglia GABAergic neurons differentiate schizophrenia from bipolar disorder,” Schizophr Res. 91(1-3):51-61 (March, 2007); Rice et al., “Critical periods of vulnerability for the developing nervous system: evidence from humans and animal models,” Environ Health Perspect., 108 (Suppl 3): 511-533 (June, 2000)). Consequently, Metabolic Enhancement Therapy directed toward balanced synthesis of GABA is clearly desirable in the autistic population. Glutamine is a common precursor for the biosynthesis of both L-glutamate and (GABA) neurotransmitters in glutamatergic and gabaergic neurons, respectively. The neurotransmitter GABA is synthesized from glutamate which has been formed from L-glutamine metabolized throughout the TCA cycle. Therefore, normal function of mitochondria and the TCA cycle is required for GABA synthesis. Recent discoveries of gabaergic neuron involvement in autism, and effective oral delivery of L-glutamine as a substrate convertible to GABA via mitochondria make possible the effective stimulation of GABA synthesis by Metabolic Enhancement Therapy. The inclusion of L-glutamine in the supplement compositions of the present invention may alleviate negative behavioral symptoms associated with GABA deficiencies in autism, ADD, ADHD and other neuronal disorders.

Taurine. Taurine, or 2-aminoethanesulfonic acid, is a major constituent of bile and is found in the high concentrations in brain and muscle. Taurine has been implicated in a wide array of physiological phenomena including inhibitory neurotransmission, [Militante, J. D.; J. B. Lombardini (November 2002). “Treatment of hypertension with oral taurine: experimental and clinical studies”. Amino Acids 23 (4): 381-393.], long-term potentiation in the striatum/hippocampus, membrane stabilization, feedback inhibition of neutrophil/macrophage respiratory bursts, adipose tissue regulation, and calcium homeostasis. Prematurely born infants who lack the enzymes needed to convert cystathionine to cysteine may become deficient in taurine. Thus, taurine is a dietary essential nutrient in these individuals and is often added to many infant formulas. Recent studies have shown that taurine can influence (and possibly reverse) defects in nerve impulse flow, motor nerve conduction velocity, and nerve sensory thresholds in experimental diabetic neuropathic rats. According to some animal studies, taurine produced an anxiolytic-like effect in mice and may act as a modulator or anti-anxiety agent in the central nervous system. [Kong W X, Chen S W, Li Y L, et al (2006). “Effects of taurine on rat behaviors in three anxiety models”. Pharmacol. Biochem. Behav. 83 (2): 271-6.]. Taurine appears to have multiple functions in the brain participating both in blood volume regulation and neurotransmission. In the latter context it has recently been shown to exert its actions by serving as an agonist at receptors of the GABAergic and glycinergic inhibitory neurotransmitter systems [F. Jia and N. Harrison. J. of Neuroscience, January, 2008]. As a result it can act as an inhibitory neurotransmitter for GABAergic and glycinergic neurons, augmenting GABA and glycine in this role. Taurine is highly concentrated in brain extracellular fluid, second only to glycine as a circulating neurotransmitter, giving further credence to the importance of taurine in achieving balanced neurotransmission. Although taurine can be synthesized from cysteine in adults, dietary consumption of meat is important to maintain the high levels of taurine required for brain and muscle function. Taurine levels were found to be significantly lower in vegetarians than in a control group on a standard American diet. Taurine does not appear to be toxic. Gram quantities can be ingested with no ill effect. In recent years, taurine has become a common ingredient in energy drinks, although it has not been shown to be energy giving, but rather relaxing. As an inhibitory neurotransmitter, taurine has been used to help treat epilepsy and other excitable brain states, where it functions as a mild sedative. Research shows low taurine levels at seizure sites and its anti-convulsant effect comes from its ability to modulate excitatory nerve impulses, thereby preventing the erratic firing of nerve cells. Doses for this effect are 500 mg three times daily.

Glutamine Supplementation. Glutamine can be supplied directly by oral supplementation with the naturally occurring amino acid L-glutamine (Tapia, R. “Biochemical pharmacology of GABA in CNS.” in Handbook of Psychopharmacology, Vol. 4, eds. L. L. Iversen, S. D. Iversen, S. H. Snyder, pp 1-58 Plenum Press, New York (1975); Waagepetersen, H. S. et al. “Metabolism of lactate in cultured GABAergic neurons studied by 13C-MNR spectroscopy.” J. Cereb. Blood Flow Met. 19, 109-117 (1998); Waagepetersen, H. S. et al. “Synthesis of vesicular GABA from glutamine involves TCA cycle metabolism in neocortical neurons.” J. Neurosci. Res. 57, 342-349 (1999); Waagepetersen, H. S. et al. “The GABA paradox: Multiple roles as metabolite, neurotransmitter, and neurodifferentiative agent.” J. Neurochem. 73, 1335-1342 (1999); Wu, J.-Y. et al. “Assay methods, purification and characterization of L-glutamate decarboxylase and GABA transaminase.” in Research Methods in Neurochemistry, Vol. 5, eds. N. Marks, R. Rodnight, pp 129-177, Plenum Press, New York (1981).”).

Vitamin B6. Vitamin B6 (pyridoxine) is converted by the body to pyridoxal phosphate which is required for the synthesis, catabolism, and interconversion of amino acids. While pyridoxal phosphate dependent reactions are legion, there a few key metabolic reactions that are directly associated with symptoms of B6 deficiency. Pyridoxal phosphate is required for the synthesis of the neurotransmitters serotonin and norepinephrine, and for the synthesis of myelin required for insulation of nerves and consequent rapid neuronal transmission. These effects are thought to explain the irritability, nervousness and depression seen with mild deficiencies, and peripheral neuropathy and convulsions observed with severe deficiencies. Vitamin B6 is also required for the synthesis of heme, and deficiencies are seen in resulting anemia. B6 is also an essential component of the enzyme glycogen phosphorylase. B6 deficiency therefore plays a part in decreased tolerance of tissues to glucose circulating in the blood, a condition associated with forms of adult onset diabetes. The RDA for vitamin B6 has been set at 1.8-2.2 mg/day for a normal adult. This requirement is increased during pregnancy, lactation, for women on oral contraceptive and with age. Inadequate ingestion of B6 in infants results in clear-cut neural dysfunction such as hyperirritability ad convulsive seizures when fed mother's milk for formulas containing less that 50 mcg of vitamin B6/liter. Being a water soluble vitamin, toxicity to vitamin B6 is rare, but has been reported to induce peripheral neuropathy if doses exceed 2,000 mg/day. Several clinical trials indicate that vitamin B6, required for synthesis of serotonin from 5-hydrotryptophan, helps alleviate depression associated with premenstrual syndrome (Gunn A D G. Int J Vitam Nutr Res 1985; (Suppl 27):213-24). Clinical trials of supplementation with 5-hydroxytryptophan, a direct precursor of serotonin show only minor signs of efficacy (Van Praag H, et al. Psychiatry Res 1980; 3:75-83.; Angst J, et al. Arch Psychiatr Nervenkr 1977; 224:175-86.; Nolen W A, et al. Acta Psychiatr Scand 1988; 78:676-83.; Nolen W A, et al, Br J Psychiatry 1985; 147:16-22.; D'Elia G, et al. Acta Psychiatr Scand 1978; 57:239-52), indicating the critical need for brain L-tryptophan in brain serotonin synthesis. Taken together, human data suggest that biosynthesis of serotonin from tryptophan is limited in clinically depressed patients, that high levels of the essential amino acid tryptophan must be available, that Vitamin B6 is required for conversion of tryptophan to serotonin, that mitochondrial energy production must be optimal to satisfy the high synthesis demands for serotonin in serotonergic neurons, and that inhibition of catabolism of tryptophan to mitochondrial acetyl CoA by use of acetyl-L-carnitine are all necessary to metabolically enhance synthesis of serotonin in the human brain.

Homocysteine Metabolism (Folinic Acid, Vitamin B12, Trimethylglycine)

Recently, homocysteine metabolism in depressed patients has been implicated in the onset and progression of the disease. Several case-control studies since the 1960's have shown high prevalences of folate and vitamin B12 deficiency in depression (Alpert J E, et al. Nutrition 2000; 16:544-546). Total plasma homocysteine levels have been shown to be sensitive markers of folate and vitamin B12 deficiency, and high concentrations of homocysteine are observed in depressed patients (Bottiglieri T, et al. J Neurol Neurosurg Psychiatry 2000; 69:228-232). Because accumulation of intracellular homocysteine is toxic for neuronal mitochondrial metabolism (Inna I. et al., The Journal of Neuroscience, 2002, 22(5):1752-1762; Lindsay T, et al., Aging Cell, 2004, 3 (2), 71-76; Baydas G, et al., Neuroscience. 2005; 135(3):879-86.), active removal of homocysteine is imperative to achieve normal mitochondrial metabolism required for the adequate biosynthesis of serotonin and other neurotransmitters necessary for normal, balanced neuronal activity.

Folinic Acid, Vitamin B12. In normal brain, homocysteine is either converted back to methionine via methionine synthase, requiring folate and B12 as co-factors, or it can be metabolized to generate the amino acid cysteine, which requires vitamins B6 and folate. The latter metabolic pathway also improves mitochondrial metabolism and ATP synthesis by producing alpha-ketoglutarate for TCA cycle use and glutathione to keep mitochondrial functions optimized via a reducing environment. Due to unknown pathological factors, removal of homocysteine is impaired in clinically depressed patients. Consequently, clinical studies designed to overcome accumulation of homocysteine and disruption of the methionine-homocysteine cycle have been successful in treating depression. Oral delivery of S-adenosyl methionine (SAMe), the intermediate in the methionine-homocysteine cycle required for all methylation processes in cells, has also been demonstrated to be an effective treatment for depression in clinical trials (Bell K M, et al. Acta Neurol Scand 1994; 154(suppl):15-8.; Bressa G M. Acta Neurol Scand 1994; 154(suppl):7-14.; Salmaggi P, et al. Psychother Psychosom 1993; 59:34-40), and depression symptoms associated with deficiency of vitamin B12 were responsive to B12 supplementation (Lindenbaum J, et al. N Engl J Med 1988; 318:1720-8.; Penninx B W, et al. Am J Psychiatry 2000; 157:715-21.). Accordingly, the supplements of the present invention are also intended to assist in the inhibition of homocysteine accumulation as a requirement for mitochondrial health and consequent optimal energy (ATP) production. A further benefit of conversion of homocysteine to glutathione in brain mitochondria is the longer term protective effects of the reductant glutathione in reducing reactive oxygen species (ROS) in mitochondria, thereby eliciting persistent protection of mitochondrial function and anti-aging effects on overall brain metabolism.

Trimethylglycine. Trimethylglycine (TMG), which derives from dietary choline, transfers one of its three methyl groups to homocysteine, converting it into methionine, which is then converted to S-adenosylmethionine, a crucial metabolite responsible for all nearly all methylation reactions required by DNA and proteins. In the course of this reaction dimethylglycine (DMG) is formed, which is a precursor for the generation of glycine and serine. Glycine or serine can pass into the brain, where serine is converted back to glycine to promote regulation of NMDA receptors. By this reasoning, supplemental use of glycine at levels of 1 to 3 grams per day has been shown to be useful in the treatment of certain forms of bipolar depression. Because the brain utilizes 10× more glycine than can be provided in a normal diet, Metabolic Enhancement Therapy using TMG in conjunction with folinic acid and vitamin B6 is a powerful stimulant for producing adequate amounts of brain glycine in individuals with compromised glycine neuroinhibitory function.

Brain Serotonin Synthesis. (L-Tryptophan, L-Acetyl Carnitine, Vitamin B6)

Anxiety, fear and panic are common manifestations in many behavioral and emotional disorders, including autism, schizophrenia, bipolar disorder and attention deficit disorder (ADD). The amygdala is intimately related to the neurobiological mechanisms that underlie emotional behavior, especially anxiety and fear. The amygdala receives sensory information from all sensory modalities, assesses the emotional significance of this information, and orchestrates an appropriate behavioral response (LeDoux, J. E., “Brain mechanisms of emotion and emotional learning,” Current Opinion in Neurobiology, 2, 191-198 (1992)). Sensory input to the amygdala can result in immediate activation of neuroendocrine and autonomic processes via efferent pathways to the hypothalamus and brain stem (Davis et al., “Neurotransmission in the rat amygdale related to fear and anxiety,” Trends in Neurosciences, 17, 208-214 (1994)). This immediate emotional response takes place before there is an opportunity to analyze whether the threat is real or perceived. A second neural circuit relays information from the amygdala to the cortex, where the frightening stimulus is analyzed in detail, and a message sent back to the amygdala to allow for modulation of the emotional response. Thus, a normal emotional response to fearful stimuli is tempered by inhibitory input from the cortex to the amygdala. Deficiencies in this cortico-amygdala pathway can result in an inappropriate emotional response, and result in panic. Panic is a heightened stage of anxiety and fear feeding itself in a positive feedback loop and jumping to faulty conclusions, that focus on impending danger and harm to the individual. Unfortunately, the inhibitory neural connections from the cortex to the amygdala are less well developed than are excitatory connections from the amygdala to the cortex. Once an emotion has been turned on, it is difficult for the cortex to turn it off, leading to a feeling of anxiety, fear or even severe panic that can render the individual incapable of normal function. Two forms of fear have been correlated with signals passed in the amygdala-cortex axis. These are spontaneous fear responses and memory conditioned fear responses. Memory conditioned fear can be modulated by the inhibitory neural transmitter, glycine, while spontaneous fear reflexes are modulated by serotonin.

L-Tryptophan. Tryptophan is an essential amino acid that plays important roles in several metabolic pathways, such as the synthesis of 5-HT. Serotonin containing neurons have cell bodies in the midline raphe nuclei of the brain stem and project to portions of the hypothalamus, the limbic system, the neocortex and the spinal cord. Serotonin is synthesized by conversion of L-tryptophan into 5-hydroxytryptamine (5-HT) and on to serotonin (FIG. 1). Given the presence of 5-hydroxytryptamine (5-HT) receptors in the amygdala (Radj a et al., “Autoradiography of serotonin receptor subtypes in the central nervous system,” Neurochem. Int., 18, 1-15 (1991)) and the general role of serotonin in depression and anxiety (Curzon, G., “Serotonergic mechanisms of depression,” Clin. Neuropharmacol., 11 (Suppl 2), S11-S20 (1988)), it was determined that 5-HT modulates the processing of sensory information within the amygdala (Stutzmann G E, et al. The Journal of Neuroscience, 1998, 18:9529) and thus may regulate amygdala-related functions. Extensive evidence suggests that because tryptophan is the only source for serotonin and melatonin in the brain (Fernstrom J D, et al. Science 1971; 173: 149-152, Fernstrom J D, et al. Science 1971; 174: 1023-1025), deficiencies in utilization of tryptophan can lead to depression and other psychological disturbances, such as anxiety, insomnia, fatigue, inability to mentally focus and low self-esteem (Van Praag H M, et al. Nutrition and the Brain, vol. 7, R J Wurtman, J J Wurtman, eds. New York: Raven Press, 1986). Because animal models of depression are severely limited in their ability to adequately assess these behaviors, therapeutic testing of tryptophan conversion to serotonin effects on depression can be reliably tested only in human subjects. Crucial to the conversion of tryptophan to 5-HT is cofactor Vitamin B6. Since these reactions all take place in serotinergic neurons in the brain, a further complication is transport of tryptophan across the blood brain barrier. Since tryptophan and phenyalanine use the same blood brain barrier transport carrier, tryptophan can be competitively excluded from brain by phenylalanine, found in high concentration in diet drinks. High serotonin levels must be maintained in brain, since intracellular serotonin in serotonergic neurons is packaged into vesicles and released at presynaptic terminal as the inhibitory NT, while serotonin secreted from these neurons into the cerebral spinal fluid bathes the brain to act as factor to support the growth and maintenance of developing serotonergic neurons. A third function of serotonin is conversion to melatonin (5-methoxy-N-acetyltryptamine), a pineal hormone (Lerner A B, et al. J Am Chem Soc 1958; 80: 2587) involved in the induction of sleep (Sugden D. Experientia 1989; 45: 922-931; Namboodiri M A., Methods Enzymol. 1987; 142, 583-590; (Sugden, D., Cena, V., and Klein, D.C. (1987) Methods Enzymol. 142, 590-596) (FIG. 1). As a consequence of these crucial roles in behavior modulation, depleted serotonin synthesis has far reaching effects on anxiety and fear modulation, neuronal development, eating and sleep disorders. Accordingly, the supplement of the present invention includes tryptophan and vitamin B6. Furthermore, acetyl-L-carnitine is also given, because optimized mitochondrial function and energy charge (ATP production) is strictly required for proper distribution of tryptophan into the serotonin synthesis pathway. Low level mitochondrial energy production and consequent deprivation of cellular ATP results in tryptophan conversion to aminomuconate-semialdehyde, which is ultimately directed toward formation of mitochondrial acetyl-CoA. Consequently, in energy deficient serotonergic neuron mitochondria, synthesis of serotonin from tryptophan is aborted in favor of acetyl CoA and ultimate ATP production. Administration of acetyl-L-carnitine in the formulation disclosed here insures sufficient acetyl CoA synthesis to direct 1-tryptophan directly into serotonin synthesis.

Brain Glycine Synthesis (Folinic Acid, Trimethyglycine, Vitamin B12, Vitamin B6)

The brain uses 10 times more glycine than can be obtained from the diet. Metabolic pathways dedicated to glycine synthesis in brain are known to be essential for normal brain function. Glycine acts as an inhibitor (antagonist) of excitatory neurons, and as a stimulant (agonist) for inhibitory neurons. Consequently, high circulating levels of glycine in the cerebral spinal fluid must be maintained to achieve balanced neuronal activity, a hallmark functional deficiency in autistic, schizophrenic, and bipolar patients. A further corrective use of glycine involves modulation of amygdala activity in extinction of conditioned fear. Extinction is an active form of learning that competes with the fear response elicited by a conditioned stimulus. Recent work suggests that the amygdala learns to extinguish an initial fear response by inhibitory signals received from the cortex and sent along glutaminergic neurons by a NMDA receptor-dependent mechanism (Davis et al., Annals NY Acad Sci 985:263-272, 2003). Since NMDA receptor antagonists disrupt extinction of conditioned fear (Davis, Eur J Neurosci 16:395, 2002), NMDA agonists (stimulation of NMDA glutaminergic neurons) should inhibit conditioned fear. However, glutaminergic neuron NMDA receptor activation with agonist drugs is associated with neurotoxicity, eliminating direct activation of glutaminergic neurons as a therapeutic. Fortunately, glutaminergic neurons can also be stimulated by glycine binding to the NMDA receptor. Glutaminergic inhibition of the amygdala can occur by binding glycine or glycine homologs to the glycine site of the NMDA receptor on glutaminergic neurons connecting the cortex to the amygdala. By this reasoning, D-cycloserine, a drug that binds the NMDA glycine site has been shown to augment the extinction of conditioned fear (Walker et al., J Neurosci 22:2343-2351, 2002). Clinical trials are ongoing with D-cycloserine in subjects with autism. In contrast, the supplements of the present invention may stimulate synthesis of the naturally occurring brain metabolite, glycine, to promote binding of glycine itself to the glycine site of the NMDA receptor, thereby stimulating glutaminergic neuron extinction of conditioned fear. For this effect, large quantities of glycine must be synthesized by the naturally occurring pathways in brain that convert trimethylglycine (TMG) to glycine.

Folinic Acid and Vitamin B12. TMG, folinic acid and vitamin B12 are administered to stimulate brain glycine synthesis through the homocysteine metabolic pathway. All of the autistic and schizophrenic patients treated with these natural metabolites have shown remarkable recoveries from persistent anxiety, fearful panic attacks, echolalia, depression and angry outbursts. These effects are believed to be due to successful modulation of amygdala function, and to reductions in homocysteine levels in these patients elicited by the disclosed formulation.

In another embodiment, the supplement further comprises at least one additional ingredient such as lithium orotate, vitamin C, vitamin D, vitamin E, vitamin B2 (riboflavin), vitamin B3 (niacin), Nystatin, vitamin B5 (pantothenic acid), calcium, and magnesium.

Lithium Orotate. Both lithium and orotate are essential nutrients. Orotate is an obligate intermediate in the de novo biosynthesis of the pyrimidine nucleotides, uridine and cytodine, from the amino acids glutamine and aspartic acid. When bound to cations such as lithium, it also serves to facilitate rapid ion transport across the blood brain barrier. Delivery of lithium to brain is therefore increased over 100 fold by complexing with orotate, dramatically reducing the effective therapeutic dose of lithium required for normal neural function. Furthermore, lithium is needed to transport folate and vitamin B12 into the brain, implicating the severe lithium deficiency found in autistic children with the requirement for high doses of these vitamins in the autistic population. However, it is imperative to recognize that only very small amounts of lithium are essential for mental health. Doses of lithium between 150-400 mcg/day have proved effective in reducing violent crime, paranoia, suicide, drug abuse and depression. Nutritional use of lithium is completely safe, in stark contrast to the extremely high doses of lithium carbonate (1-2-grams/day; 10,000-20,000 mcg/day) used as a prescribed drug to treat schizophrenia, depression, obsessive-compulsive and other neural disorders. Because of the poor absorptive properties of lithium carbonate through the blood brain barrier, excessive doses of lithium carbonate are required in order to pass microgram quantities of lithium into the brain, leaving toxic amounts of lithium in the blood and body tissues. Because of its highly effective transport into brain, lithium orotate provides a safe and non-toxic vehicle for delivery of lithium to brain without deleterious side effects. A 12 mg dose of lithium orotate contains 520 mcg of lithium available to the brain, well below the threshold amount required for deleterious side effects, but sufficient to provide an effective nutritional supplement within the provisional Recommended Daily Allowance (RDA) for a 70 kg adult of 1,000 mcg/day. [References: Moore, G J, et al. Lithium-induced increase in brain grey matter. Lancet. 2000, Vol. 356, pp. 1241-42.; Schrauzer, G N. Lithium; occurrence, dietary intakes, nutritional essentiality. J Am Coil Nutr. 2002, Vol. 21, pp 14-21.; Schrauzer G N. et al. Lithium in scalp hair of adults, students and violent criminals. Effects of supplementation and evidence for interactions of lithium with Vitamin B and other trace elements. Biological Trace Element Research, 1992, Vol. 34, pp 161-76.; J B Adams, et al. Analyses of toxic metals and essential minerals in the hair of Arizona children with autism and associated conditions, and their mothers. Biological Trace Element Research. 2006, Vol. 110, pp 193-209.]

Vitamin B2 (Riboflavin). In the body riboflavin is converted to the coenzymes favin adenine dinucleotide (FAD) and flavin mononucleotide (FMN), both of which are involved in a wide variety of oxidation-reduction reactions. The flavin coenzymes are essential for energy production in the mitochondria. A key regulatory enzyme of the TCA cycle, pyruvate dehydogenase, requires the coenzyme FAD. The recommended riboflavin intake is 1.2-1.7 mg/day for normal adults. Because milk, meat, eggs, bread and cereal products are rich in riboflavin, deficiencies are quite rare in the U.S. However, individuals on self-restricting diets such as chronic alcoholics, or those with mal-absorption problems, including many autistic children may be subject to riboflavin deficiency.

Vitamin B3 (Niacin). Niacin and niacinamide are both converted to the essential oxidation-reduction coenzymes NAD and NADP in the body. Although niacin can be synthesized from tryptophan in the presence of vitamin B1, B2, and B6, the process is extremely inefficient, especially on a marginal diet. Pronounced deficiencies lead to pellagra which is characterized by dermatitis, diarrhea and dementia. Neurologic symptoms are associated with actual degeneration of nervous tissue. Because of food fortification, pellagra is rare, being primarily seen in alcoholics, patients with severe malabsorption problems, and the elderly on very restricted diets. The current RDA for niacin is 20 mg free niacin/day. If used in pharmacologic doses (2-4 g/day), it can cause a number of metabolic effects not related to normal function of the vitamin. These include vasodilation (flushing reaction), a marked decrease in circulation fatty acids, cholesterol and lipoproteins, and an elevation of serum glucose and uric acid. Used as a nutritional supplement in lower doses of up to 50 mg/day, it is completely safe.

Vitamin B5 (Pantothenic Acid). Panthothenic Acid is an essential component of coenzyme A (CoA) and acyl carrier protein (ACP) and thus is required for the metabolism of all fat, protein, and carbohydrate via the TCA cycle. More than 70 enzymes have been described to date that utilize CoA or ACP derivatives. However, pantothenic acid deficiencies do not appear to be a serious concern, probably because pantothenic acid is very widespread in natural foods, and most symptoms of vitamin B5 deficiency are vague and mimic those of other more common B vitamin deficiencies. A RDA of 10 mg/day has been established.

Vitamin D, Calcium and Magnesium. Both vitamin D and calcium are obligatory to support bone growth and remodeling. The requirement is especially critical for growing children. Vitamin D can be produced in the skin by ultraviolet radiation of a metabolite of cholesterol, or by ingestion of natural sources of vitamin D in fish, liver and egg yolk. Milk products are also routinely fortified with vitamin D. Individuals with allergies to milk products (lactose and/or casein intolerance), who exhibit fat malabsorption, are on low cholesterol diets, or are sunlight sensitive should be supplemented with vitamin D and calcium. Although a RDA of 400 IU has been established for vitamin D, higher amounts may be necessary for autistic children subject to deficiencies. However, deleterious side effects have been observed with pharmacologically administered extreme high doses. Calcium is the most abundant mineral in the body, essential for bone growth, enzyme function, hormonal responses, muscle contraction and blood coagulation. An RDA of 1000 mg/day has been set, with an additional 400 mg allowance for pregnant and lactating women. Dietary surveys indicate that a significant portion of low income children and the elderly do not have adequate calcium intake. This may be extended to autistic children who encounter milk allergies or refuse to consume sufficient quantities of meat. Magnesium is also ubiquitous in living tissue and is required for many enzymes function and for neuromuscular transmission. Symptoms of magnesium deficiency are muscle weakness, tremors, and cardiac arrhythmia. Consequently, supplementation with magnesium is recommended. A RDA of 385 mg/day has been established.

Nystatin (prescription). Nystatin is an antifungal antibiotic obtained from Streptomyces noursei. It is both fungistatic and fungicidal against a wide variety of yeasts and yeast-like fungi, but exhibits no activity against bacteria, protozoa or viruses. Gastrointestinal absorption of Nystatin is insignificant, with orally administered nystatin passing unchanged in the stool. It is indicated for treatment of candidiasis and a variety of yeast infections occurring in the gastrointestinal tract. It is generally well tolerated by all age groups, even during prolonged use.

Vitamins C and E. Vitamins C and E are extremely well known reductants that serve to control damaging reactive oxygen species (ROS); vitamin E in cell membranes, and vitamin C in the cell cytoplasm and mitochondria. Control of oxidation in mitochondrial membranes and matrix is crucial for prolonged mitochondrial function. The constant source of electrons bleeding off of the electron transport chain in the inner mitochondrial membrane during generation of ATP quickly react with the oxygen present to produce highly reactive ROS that degrade enzymes and the mitochondrial membrane. Mitochondria use large quantities of glutathione synthesized from cysteine, together with ingested Vitamin C from the diet to prevent formation of ROS during electron transport. Vitamin E is also supplied from the diet, and serves to protect all cell membranes from oxidative degradation. Vitamin C also has an crucial biological role in a number of hydroxylation reactions in the body, and is especially important for normal functioning of connective tissue, bone, the adrenal glands, liver, and the immune system. A RDA of 60 mg/day of Vitamin C has been established, but a number of studies indicate that much higher doses (10-20×RDA) are beneficial and well tolerated. A RDA of 30 I.U./day (International Units=mg) has been established for Vitamin E, although supplementation of up to 400 I.U. is now recommended. Because vitamin E is fat soluble, it has the potential for toxicity. However, no instances of toxicity have been reported at doses of up to 800 mg/day.

The supplement can be provided as a liquid, as a water-based solution, or as a powder which can be made into a liquid by adding water or any other physiologically acceptable liquid to the powder. In other embodiments, the powder may be in a form suitable for adding to food. In powder form, the supplement may be sprinkled on food or directly mixed in with food.

The supplements of the present invention are preferably in liquid form suitable for oral administration and are delivered orally in order to significantly enhance absorption in subjects with compromised intestinal absorptive capacity, such as autistic and schizophrenic patients.

In some embodiments, it may be desirable to provide both a powder and a liquid form, for example, for shipping and/or storage purposes, it may be desirable to provide the supplement of the present invention as a powder containing the ingredients with a liquid such as distilled water that can be combined by the user at the time of use.

Formulations containing the supplements compositions of the present invention may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

In one preferred embodiment, the supplement is in a liquid formulation that is suitable for oral administration. Such formulations can be prepared so they are self-administered or administered by a caregiver.

In certain embodiments, the supplement of the present invention may be provided in formulations used in the pharmaceutical arts. Such embodiments include, but are not limited to, capsule forms, sustained-release gels, patches, and forms suitable for injection.

In one embodiment, the supplement is administered in the form of an injection via subcutaneous or intramuscular injection.

The nutritional supplements of the present invention may additionally comprise conventional carriers, adjuvants or diluents. The following formulation methods and excipients are merely exemplary and in no way limit the invention.

The nutritional supplements according to the present invention can be provided as a compositions containing carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The supplements of the invention may be formulated so as to provide quick, sustained or delayed release of the active ingredient after their administration to a subject by employing any of the procedures well known in the art.

For example, the compositions of the present invention can be dissolved in oils, propylene glycol or other solvents which are commonly used to produce an injection. Suitable examples of the carriers include physiological saline, polyethylene glycol, ethanol, vegetable oils, isopropyl myristate, etc., but are not limited to them. For topical administration, the compounds of the present invention can be formulated in the form of ointments and creams.

In a sustained-release gel, the supplement may be combined with a suitable gelling agent and can be applied to the skin or mucosal lining of the buccal cavity to facilitate absorption by the body. The sustained release gel can comprise any of the gels known in the art for this purpose. In one embodiment, the gel may be a gelling agent comprising a heteropolysaccharide gum and a homopolysaccharide gum capable of cross-linking when exposed to a body fluid. The gel can also include an inert pharmaceutical diluent, if desired.

The supplements of the present invention may also be provided in the form of a patch. The patch can be either a single-layer drug-in-adhesive or multi-layer drug-in-adhesive. The patch can include filler materials included as components in a plasticizer/polyisobutylene adhesive matrix. As an example, a monolithic adhesive drug-containing layer can include an adhesive system of polyisobutylene and a plasticizer. The polyisobutylene may be a blend of a high molecular weight polyisobutylene and a low molecular weight polyisobutylene. In manufacturing these compositions, it is preferable to use a solvent for the polyisobutylene which is a non-solvent for the drug. The plasticizer which is utilized in conjunction with polyisobutylene to form the adhesive layer is a generally insert, organic, apolar, nonvolatile hydrophobic liquid. In particular, the plasticizer may be a hydrophobic liquid in which the components of the supplement of the present invention are moderately soluble. The patch can include a filler in the mixture of plasticizer and polyisobutylene. Such fillers include a number of inert filler components including metal oxides, inorganic salts, synthetic polymers, clays and the like. Examples of metal oxides include zinc oxide, magnesium oxide, titanium oxide, and calcium oxide. Examples of inorganic salts include calcium, magnesium and sodium carbonate, calcium and magnesium sulfate, calcium phosphate, and the like. Examples of synthetic polymers include methacrylic resin, nylon, polyethylene, and the like. The clay compounds include talc, bentonite and kaolin.

In a preferred method, the supplement is orally administered in an amount and for a time period sufficient to treat a given condition.

The desirable dose of the composition varies depending on the condition and the weight of the subject, severity, form of the supplement, route and period of administration, and may be chosen by those skilled in the art. Any suitable amount of the composition of the invention may be administered and dosage levels will vary according to the nature of the disease to be treated or prevented and the subject.

The supplements of the present invention are preferably administered to mammals, in particular, humans. The supplements of the present invention can be administered to a mammal via various routes. All modes of administration are contemplated, for example, administration can be made orally, rectally or by intramuscular, subcutaneous, intracutaneous, intrathecal, epidural. In preferred embodiments, the composition is administered orally.

As described above, the nutritional supplements of the present invention may be used to normalize inhibitory and excitatory neuron function. Based in part on this observation, the formulations of the present invention may be used to treat a variety of conditions. In one method, a nutritional supplement comprising acetyl-L-carnitine, α-lipoic acid, biotin, vitamin B1, L-glutamine, Vitamin B6, taurine, folinic acid, vitamin B12, trimethylglycine, and L-tryptophan is administered to a mammal, preferably a human, to normalize their inhibitory and excitatory neuron function.

As illustrated in FIG. 1, mitochondrial metabolism is fundamentally related to cellular energy levels. Accordingly, another method of the present invention modifies mitochondrial metabolism. This method comprises administration of a nutritional supplement comprising acetyl-L-carnitine, α-lipoic acid, biotin, vitamin B1, L-glutamine, Vitamin B6, taurine, folinic acid, vitamin B12, trimethylglycine (betaine), and L-tryptophan to a mammal, preferably a human, to modify their mitochondrial metabolism.

As described above, homocysteine metabolism in depressed patients has been implicated in the onset and progression of depression. Accordingly, in certain situations, it may be desirable to regulate homocysteine. Accordingly, another method of the present invention is used to prevent cellular accumulation of homocysteine. In this method, a nutritional supplement comprising acetyl-L-carnitine, α-lipoic acid, biotin, vitamin B1, L-glutamine, Vitamin B6, taurine, folinic acid, vitamin B12, trimethylglycine, and L-tryptophan is administered.

The present invention also provides a method for increasing metabolic production of serotonin, glycine and GABA, comprising administration of acetyl-L-carnitine, α-lipoic acid, biotin, vitamin B1, L-glutamine, Vitamin B6, taurine, folinic acid, vitamin B12, trimethylglycine (betaine), and L-tryptophan.

The present invention also provides methods for treating a mammal having a disease or condition, including, but not limited to, acute anxiety, hyperactivity, panic attacks, confusion, involuntary movements, a sleep disorder, a mood disorder, depression, anger, seizures, obsessive-compulsive behavior, unregulated fear, panic, or attention deficit disorder. To treat these diseases or conditions, a nutritional supplement including acetyl-L-carnitine, α-lipoic acid, biotin, vitamin B1, L-glutamine, Vitamin B6, taurine, folinic acid, vitamin B12, trimethylglycine (betaine), and L-tryptophan is administered.

The present invention also includes a method for treating autism, schizophrenia attention deficit disorder (ADD), attention deficit hyperactive disorder (ADHD) and neuronally based immunologic disorders comprising administration of a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.

The present invention also provides a method for treating physical hyperactivity, involuntary movements, low motile bowel syndrome, or intestinal malabsorption comprising administration of a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.

Another embodiment of the present invention is intended for administration to children and is described in the Example section below. In particular, a nutritional supplement for administration to a child includes acetyl-L-carnitine, α-lipoic acid, biotin, vitamin B1, L-glutamine, Vitamin B6, taurine, folinic acid, vitamin B12, trimethylglycine, L-tryptophan, vitamin B5 (pantothenic acid), vitamin B3 (niacin), and vitamin B2 (riboflavin).

### EXAMPLES

### Examples of Formulations

### Example 1

### Standard Supplement

A supplement according to the present invention may be prepared according to the table below:

SUPPLEMENT TABLE.

| Component | | | Avg. Daily | | | |
|---|---|---|---|---|---|---|
| Dose | Concentration | | Dose (20 ml) | | Range Daily | |
| Acetyl-L-Carnitine | 25 | mg/5 ml | 100 | mg | 10-1000 | mg |
| α-Lipoic acid | 25 | mg/5 ml | 100 | mg | 50-500 | mg |
| Biotin | 37.5 | mcg/5 ml | 150 | mcg | 50-300 | mcg |
| Vitamin B1 | 3 | mg/5 ml | 12 | mg | 1.5-100 | mg |
| L-Glutamine | 250 | mg/5 ml | 1000 | mg | 20-3000 | mg |
| Vitamin B6 | 6.25 | mg/5 ml | 25 | mg | 2-100 | mg |
| Taurine | 125 | mg/5 ml | 500 | mg | 100-3000 | mg |
| Folinic Acid | 250 | mcg/5 ml | 1000 | mcg | 400-4000 | mcg |
| Vitamin B12 | 7.5 | mcg/5 ml | 30 | mcg | 6-200 | mcg |
| Trimethylglycine | 625 | mg/5 ml | 2500 | mg | 500-3000 | mg |
| L-Tryptophan | 250 | mg/5 ml | 1000 | mg | 20-4000 | mg |

Daily Dosage. The formulation is administered as an oral liquid containing all of the components in purified water. A total daily administration of from 5 to 40 ml may be used, depending on individual subject need. Multiple individual doses of 5 ml can be given once a day, 2×/day at 8 hour interval, 3×/day at 6 hour intervals, 4×/day at 4 hour intervals or 8×/day at 2 hr intervals, depending on need. The formula may include other inactive ingredients for flavor, osmotic stability, pH stability, and preservation. Additional supplements may be administered as needed. These may include, but are not restricted to: lithium orotate, Nystatin (prescription), vitamins B2, B5, C, E, D, calcium and magnesium. Average recommended daily doses of 4×5 ml deliver approximately 100 mg/day of acetyl-L-carnitine, 1,000 mg/day of L-tryptophan, 100 mg/day α-lipoate, 150 mcg/day biotin, 25 mg/day of vitamin B6, 30 mcg/day of vitamin B12, 2,500 mg/day of trimethylglycine, 1,000 mg/day of L-iglutamine, 1000 mcg/day of folinic acid, 12 mg/day of vitamin B1, and 500 mg/day of taurine. The range of daily dosage for each component may deviate from the recommended dose depending on individual need. Approximate range values for each component are given in the Supplement Table.

### Example 2

### Children's Supplement

The supplement described below is designed for prevention or relief of symptoms associated with development of early childhood neuronal disorders, including but not limited to autism, ADD, ADHD, obsessive compulsive behavior, depression, mania, anxiety, panic, and lassitude. The supplement is intended to be administered as the child is weaned from baby formula, typically from 1-2 years of age. The supplement includes:

### Children's Supplement

| Component | Concentration | |
|---|---|---|
| Acetyl-L-Carnitine | 25 | mg/5 ml |
| α-Lipoic acid | 1.25 | mg/5 ml |
| Biotin | 7.5 | mcg/5 ml |
| Vitamin B1 | 1.25 | mg/5 ml |
| L-Glutamine | 50 | mg/5 ml |
| Vitamin B6 | 1.25 | mg/5 ml |
| Taurine | 50 | mg/5 ml |
| Folinic Acid | 50 | ug/5 ml |
| Vitamin B12 | 1.5 | mcg/5 ml |
| Trimethylglycine | 125 | mg/5 ml |
| L-Tryptophan | 50 | mg/5 ml |
| Pantothenic Acid | 1.25 | mg/5 ml |
| (vitamin B5) | | |
| Niacin | 1.25 | mg/5 ml |
| (vitamin B3) | | |
| Riboflavin | 1.25 | mg/5 ml |
| (vitamin B2) | | |

Dosage is normalized to a body weight of approximately 20-50 lbs. Formula is supplied as an orally administered containing all components in purified water. The formula may include other inactive ingredients for flavor, osmotic stability, pH stability, and preservation. Additional supplements may be administered as needed. These may include, but are not restricted to: lithium orotate, vitamins C, E, D, calcium and magnesium. Administration is anticipated to be 4-8 times daily. This formulation is designed to sustain supplementation received in baby formula and to metabolically enhance specific biosynthetic pathways required for normal production of tricarboxylic acid cycle derived ATP, acetyl CoA, β-oxidation derived ATP, and production of the inhibitory neurotransmitters glycine, serotonin, and GABA.

### Case Studies of Therapeutic Efficacy.

### Example 1

Autistic 15 year old male diagnosed with pervasive developmental disorder not otherwise specified (PDD-NOS) at age 6. Subject's behaviors were noteworthy for social withdrawal, inability to engage, attend or converse with others, obsessive compulsive behavior and perservative involuntary movements, especially pacing. Subject's intellectual and academic abilities are in the average range. However, cognitive abilities were not readily recognized because of maladaptive behaviors. Most noteworthy was a profound lack of energy, characterized by an inability to stand for even one minute, an inability to keep his head up while in class and complaint of constant fatigue. The subject's appetite was poor, food choices were limited and he was extremely thin. Prior to beginning Metabolic Enhancement Therapy, subject was on several prescription medications, Adderall, Risperdal and Prozac. Following a precipitous physiologic decline, medications were slowly reduced and eventually eliminated. No improvement in behaviors, involuntary movements, sleep habits, or general energy levels resulted from withdrawal of medications. Following a 14 day interval in complete absence of medication, subject was started on the disclosed formulation. Within a few days of administration, his condition improved markedly. His rapid response was significant for elevated mood, increased energy, reduction in obsessive compulsive behavior, increased appetite, ease of falling asleep and easily aroused in the morning. Overall, his response to the disclosed formulation has been remarkably superior to that of all prescribed medications or to the absence of medications. No adverse side effects have been noted after 6 months of continual Metabolic Enhancement Therapy with the disclosed formulation.

### Example 2

16 year old male diagnosed with moderately functioning autism at age 2. Subject's behaviors were noteworthy for acute social withdrawal, pronounced language deficit, obsessive compulsive behaviors, acute anxiety attacks, aggression, hyperactivity, loss of concentration, fear and panic disorder, hyperactivity, depression, sleep deficit and acoustic hypersensitivity. Subject experienced loss of appetite and was underweight, although growth and sexual development was not stunted. Intestinal disorders included lactose and gluten intolerance from age 2 to 8 years, low motile bowel syndrome from approximately 10 years of age, chronic intestinal yeast infections with resulting psychologic disturbance. Subject appeared to be cognitively intact with excellent reading and spatial organization skills, and possessed of warm and friendly social interactions when not sensory overwhelmed. Medication with secretin, amoxtatine, serotonin uptake inhibitors and megavitamins proved totally ineffective in alleviating behavioral symptoms. Nystatin effectively controlled yeast infections. Treatment with components of the disclosed formulation beginning at age 12, and continued administration of the complete disclosed formulation from the age of 14 resulted in immediate and remarkable modulation and diminution of the behavioral disorders described, including alleviating symptoms of acute anxiety, obsessive compulsive behaviors, agoraphobia, panic attacks, aggressions, depression, and sleep disorder. Language deficits have gradually improved. Subject demonstrated remarkable mood elevation, self-confidence, improvement in scholastic performance, and self-modulation of behaviors, including aggressiveness and hyperactivity when treated with Metabolic Enhancement Therapy. No adverse side effects have been observed after 4 years of continual therapy with the disclosed formulation.

### Example 3

52 year old male diagnosed with schizophrenia in his early 20s. Hospitalized several times suffering from acute psychotic attack with hallucinations and paranoia. Treated with many typical and atypical antipsychotic drugs. The antipsychotic neuroleptic medications suppressed overt psychotic thoughts but did not alleviate “negative” symptoms. Over the years, several diagnoses were given, including bipolar disorder, obsessive-compulsive disorder, affective disorder, and combinations thereof. Medications have included lithium, olanzapine, prozac, and several sleep medications. None provided relief of symptoms, but did induce tremors, rigidity, weight gain and diabetes. Patient went through years of treatment programs and weekly psychiatric sessions. Although he managed (with extreme difficulty) to obtain BA and MA degrees, paranoia and anxiety prevented him from working. Recent history. Patient has been on an atypical antipsychotic (clozapine) and high doses of lithium carbonate for the past 2 years. Obsessive-compulsive behavior progressively deteriorated during this time. Patient started on Metabolic Enhancement Therapy with the disclosed formulation, while remaining on clozapine and lithium. Within 3 weeks, the patient dramatically increased in the ability to focus and concentrate at reading and writing, eliminated hyperactive movements (pacing), dramatically reduced anxiety and obsessive compulsive behavior, demonstrated significantly improved judgment, greatly increased levels of confidence and interest in intellectual work and social interactions. Subject has experienced a general increase in energy level and can now walk more than 2 miles a day. Patient has dramatically progressed in his ability to function as a teacher and activities administrator at his therapeutic clubhouse. Patient's psychiatrist has noted his remarkable recovery from long-term schizophrenia, and has begun reduction of lithium and clozapine medications.

### Example 4

50 year old female diagnosed with acute anxiety disorder and depression. Patient treated for 2 years with Risperdol and Prozac. Subject experienced minimal effectiveness in reducing anxiety, and experienced negative side effects associated with the medications. While still taking the prescribed medications, patient began Metabolic Enhancement Therapy with the disclosed formulation without L-tryptophan or L-glutamine. Subject reported dramatic reduction in anxiety and associated stress within 1 week of starting the course of treatment. Persistent improvement and sense of well-being prompted patient to begin programmed reduction of Risperdol and Prozac dosage, with the aim of eliminating medication in favor of continued treatment with the disclosed formulation.

### Example 5

14 year old female diagnosed with high functioning autism at age 6. Subject exhibited good language ability and social skills, but demonstrated pervasive anxiety and panic attacks initiated by conditioned fear reflex responses. Subject exhibited increased social withdrawal and behavioral difficulties in adolescence that appeared to be outside the range of normal behavior for the age cohort. A plethora of treatments, including music therapy, brain electrical stimulation, and educational behavior modification programs failed to produce beneficial effects. Patient began Metabolic Enhancement Therapy with the disclosed formulation at age 14 and reported remarkable amelioration of anxiety and panic, with concomitant significant improvement in behavior reported by subject's parents. Beneficial effects were observed after 1 week of treatment with the disclosed formulation.

Although the present invention has been described with respect to a specific preferred embodiment thereof, various changes and modifications may be suggested to one skilled in the art and it is intended that the present invention encompass such changes and modifications as fall within the scope of the appended claims.

We claim:

1. A nutritional supplement comprising: acetyl-L-carnitine, α-lipoic acid, biotin, vitamin B1, L-glutamine, Vitamin B6, taurine, folinic acid, vitamin B12, trimethylglycine, and L-tryptophan.

2. The supplement of claim 1 further comprising at least one ingredient selected from the group consisting of lithium orotate, vitamin C, vitamin E, vitamin D, vitamin B2 (riboflavin), vitamin B3 (niacin), Nystatin, vitamin B5 (pantothenic acid), calcium and magnesium.

3. The supplement of claim 1 further comprising vitamin B5 (pantothenic acid), vitamin B3 (niacin), vitamin B2 (riboflavin).

4. The supplement of claim 1 in the form of a liquid, powder, or combination of liquid and powder.

5. The supplement of claim 1 in a liquid formulation suitable for oral administration.

6. The supplement of claim 1 in the form of a capsule.

7. The supplement of claim 1 comprising a powder suitable for sprinkling on food.

8. The supplement of claim 1 dissolved in a liquid.

9. The supplement of claim 1 in a sustained release gel.

10. The supplement of claim 1 in a patch.

11. The supplement of claim 1 in a form suitable for injection.

12. A method to normalize inhibitory and excitatory neuron function in a mammal comprising administering to said mammal a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.

13. A method to modify mitochondrial metabolism comprising administering to a mammal a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.

14. A method for preventing cellular accumulation of homocysteine comprising administering to a mammal a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.

15. A method for increasing metabolic production of serotonin, glycine and GABA in a mammal comprising administering to said mammal a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.

16. A method for treating a mammal having a disease or condition selected from acute anxiety, hyperactivity, panic attacks, confusion, involuntary movements, a sleep disorder, a mood disorder, depression, anger, seizures, obsessive-compulsive behavior, unregulated fear, panic, or attention deficit disorder, comprising administering to said mammal a nutritional supplement comprising acetyl-L-carnitine, α-lipoic acid, biotin, vitamin B1, L-glutamine, Vitamin B6, taurine, folinic acid, vitamin B12, trimethylglycine (betaine), and L-tryptophan.

17. A method for treating a mammal having a disease selected from autism, schizophrenia attention deficit disorder (ADD), attention deficit hyperactive disorder (ADHD) and neuronally based immunologic disorders comprising administering to said mammal a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.

18. A method for treating physical hyperactivity, involuntary movements, low motile bowel syndrome, or intestinal malabsorption in a mammal comprising administering to said mammal a nutritional supplement comprising: acetyl-L-carnitine, α-lipoic acid, biotin, vitamin B1, L-glutamine, Vitamin B6, taurine, folinic acid, vitamin B12, trimethylglycine (betaine), and L-tryptophan.
